(19) United States
(12) Patent Application Publication
(10) Pub. No.: US 2003/0224392 A1
(43) Pub. Date: Dec. 4, 2003

(54) Method for identification of mutant antigens with enhanced immunogenicity

(76) Inventors: Manuel Engelhorn, New York, NY (US); Alan N. Houghton, New York, NY (US); Gabriele Noffz, New York, NY (US)

Correspondence Address: OPPEDAHL AND LARSON LLP, P O BOX 5068, DILLON, CO, 80435-5068, US

(21) Appl. No.: 10/353,716

(22) Filed: Jan. 28, 2003

Related U.S. Application Data
(60) Provisional application No. 60/352,706, filed on Jan. 28, 2002.

(51) Int. Cl. C12Q 1/68; G01N 33/53

(52) U.S. Cl. 435/6; 435/7.1

(57) ABSTRACT

A method is provided for identifying mutant antigens with enhanced immunogenicity that does not depend on any a priori knowledge of the structure of the native antigen. Antigens identified in this way may be used to induce an immune response to a target antigen in a subject comprising administering to the subject a vaccine composition comprising the mutant antigen corresponding to the target antigen in an amount sufficient to induce an immune response to the target antigen. The target antigen may be a self-antigen.

This application claims the benefit of U.S. Provisional Application No. 60/352,706, filed Jan. 28, 2002, which is incorporated herein by reference.

### BACKGROUND OF THE INVENTION

This application relates to a method for identifying mutant antigens (nucleic acid or peptide) which may be used to induce an immune response to the corresponding unmutated antigen.

The immune system provides a sophisticated and multi-faceted defense against antigens which are recognized as foreign. Antigens which are recognized, and which therefore stimulate an immune response, are referred to as immunogenic. However, not all antigens are immunogenic, and there are many instances of disease that the immune system deals with poorly, if at all. These include most cancers and infectious organisms such as human immunodeficiency virus-1, Mycobacterium tuberculosis, Borrelia burgdorferi (the causative organism of Lyme disease), Epstein Barr virus, papilloma virus, hepatitis viruses and cytomegalovirus (CMV) to which the immune system fails to mount an effective response. This may be due to tolerance to self-antigens (i.e. the body does not recognize a cancer cell as foreign) or because the antigen is an inherently weak immunogen. Regardless of the cause, however, these factors make the product of vaccines targeting these conditions both desirable and difficult.

Various approaches have been taken to try to overcome the inherent non-immunogenicity of cancer and other antigens. These approaches have included the identification of more immunogenic variants of the antigens of interest through modification of the epitope structure or through modification of MHC-binding regions, as well as the addition of adjuvants to enhance antigenicity. The general concept behind these prior approaches is the identification of a single species of antigen with improved immunogenicity. This can be based on structural information at the crystallographic level, information which is available for only a very limited number of human or animal MHC molecules, or extensive trial and error testing. The present invention represents a departure from this traditional approach.

### SUMMARY OF THE INVENTION

The present invention provides a method for identifying mutant antigens with enhanced immunogenicity that does not depend on any a priori knowledge of the structure of the native antigen. Antigens identified in this way may be used to induce an immune response to a target antigen in a subject comprising administering to the subject a vaccine composition comprising the mutant antigen corresponding to the target antigen in an amount sufficient to induce an immune response to the target antigen. The target antigen may be a self-antigen.

In accordance with the invention, a method is provided for identifying a mutant form of a target antigen having immunogenic properties. The method comprises the steps of

(a) creating a plurality of mutant forms of the target antigen;

(b) placing the mutant forms into a plurality of subpools, each subpool containing at least one mutant form of the target antigen;

(c) combining aliquots of the subpools into an initial set of test pools; each member of the initial of test pools comprising at least two of the subpools;

(d) testing each member of the initial set of test pools for the ability to induce an immune response to the target antigen in vivo;

(e) selecting at least one selected test pool from the initial set of test pools for further processing, said selected test pool being able to induce an immune response against the target antigen in vivo,

(f) combining further aliquots of the subpools which were included in the selected test pool to form a second set of test pools, each member of the second set of test pools comprising at least two of the subpools, and retesting each member of the second set of test pools for the ability to induce an immune response to the target antigen in vivo;

(g) performing additional cycles of selecting an effective pool from among the pools of a tested set of pools, and creating new sets of pools from the subpools making up that selected pool, until the selected pool contains only a single mutant; and

(h) characterizing the mutant form of the antigen in the single pool. The initial mutant forms created in step (a) are suitably created by a random or non-directed mutation process which gives rise to many diverse species of mutants.

### BRIEF DESCRIPTION OF THE DRAWINGS

FIG. 1 shows a pictorial representation of the first screening cycle using the method of the invention.

FIG. 2 is a partial sequence alignment between wild-type murine gp75 (wtmp75) and mutants 2C and 4G.

### DETAILED DESCRIPTION OF THE INVENTION

The present invention provides a method for identifying immunogenic mutant forms of a target antigen which can be used for inducing an immune response to a target antigen.

The target antigen against which the invention induces an immune response may be any antigen for which a therapeutic benefit is derived as a result of the induction of an immune response, including antigens associated with pathogenic microorganisms and antigens associated with cancers. The invention is particularly applicable for inducing an immune response to inherently non-immunogenic or poorly immunogenic antigens, including self antigens. Specific, non-limiting examples of target antigens include gp75/TRP-1, TRP-2, tyrosinase, gp100/pMel17 on melanoma; prostate specific membrane antigen, prostate specific antigen and prostate stem cell antigen on prostate cancers; HER2/neu and the mucin MUC1 on breast cancers; CD19 and CD20 on malignancies of B lymphocyte origin; MAGE, BAGE and GAGE, NY-ESO-1 and other “cancer-testes” antigens on a variety of cancer types; gene products from the human immunodeficiency virus-1; angiogenic factors (such as VEGF, bFGF, angiopoietins, and ELR C-X-C chemokines); tumor suppressor genes such as p53; dipeptidyl peptidase IV and fibroblast activation protein-1.

As used in the specification and claims of this application, the phrase “inducing an immune response” refers to both the stimulation of a new immune response or to the enhancement of a pre-existing immune response to a target antigen. The immune response may be a cytolytic T-cell mediated cellular immune response or a B-cell mediated humoral response, or some combination thereof.

The term “subject” refers to the living organism being treated to induce an immune response. The subject will generally be mammalian or avian. Preferred “subjects” are human patients.

Mutant forms of a target antigen identified in accordance with the method of the invention are used to induce an immune response to a target antigen in a subject by administering to the subject a vaccine composition comprising the mutant form corresponding to the target antigen in an amount sufficient to induce an immune response to the target antigen. The term “corresponding” encompasses both mutant forms of the target antigen per se (i.e, peptide vaccine molecule species) and nucleic acid vaccine molecule species encoding the mutant forms of the target antigen.

In a first embodiment of the invention, the vaccine compositions of the invention comprise a mutated nucleic acid which encodes a mutant variant of the target antigen. Unlike prior methods which focus on using single, carefully selected mutant antigens, the present invention can utilize a plurality of different species of mutant nucleic acid all derived from a starting nucleic acid encoding the target antigen by random or non-directed mutation processes. Thus, the first step of the method of the invention is creating a plurality of mutant forms of the target antigen.

To create the plurality of mutant forms, one starts with a nucleic acid sequence encoding the target antigen. This sequence may be in the form of PCR amplicon, or it may be incorporated in a vector system to facilitate its reproduction in an appropriate host. The sequence may be cDNA encoding the entire antigen or it may be a partial sequence encoding only a portion of the antigen. Although there is no absolute minimum size, partial sequences used will preferably be at least 24 bases, encoding 8 amino acids. This “starting nucleic acid sequence” is used as the starting material for generating the vaccine compositions of the invention.

The starting nucleic acid sequence may be an accepted “wild-type” sequence derived from a normal source. In this regard, it will be appreciated that polymorphic sequences may have a multiplicity of “normal” or “wild-type” sequences, and that it is not critical which of these sequences are used as the starting sequence. The starting nucleic acid sequence may also be a mutant sequence (i.e. a sequence which differs from the established norm.) The starting nucleic acid sequence may also be (but does not have to be) derived from the subject. Thus, for example, in the latter case, a subject's own cancer cells could be used as a source for the starting nucleic acid sequence.

Mutations, which can be insertions, deletions, translations, or inversions of one or more bases, can be introduced into the starting nucleic acid sequence using any of various known techniques. For example, random mutations can be introduced into the starting nucleic acid sequences using error-prone PCR as described in Cadwell et al. in PCR Methods and Applications 2:28-33 (1992) and PCR Methods and Applications 3:5136-5140 (1994). Mutations can also be introduced into the starting nucleic acid sequence by expressing the starting nucleic acid sequence in bacteria that are prone to mutations (for example Stratagene's XL 1-RED competent cells) or by exposing the starting nucleic acid to mutagenic principles such as chemicals, x-rays or ultraviolet radiation.

The result of these methods is a library of nucleic acid made up of many copies of mutated nucleic acid, with most individual nucleic acid molecules containing unique combinations of mutations. Aliquots of this library are then subcloned into an expression vector and can be used to create an “indexed library” of the mutant forms by picking clones (without required selection for immunogenicty) and placing one (or at most a few) clones into each of a plurality of subpools. Aliquots from these subpools are then combined to form an initial set test pools, each of which contains the mutant forms from two or more subpools. The test pools are tested for their ability to induce an immune response in vivo in a test animal such as a mouse, and the results of these tests are used to select one or more test pools from the initial set which are able to induce an immune response.

The next step is the creation of a second set of test pools, each containing a smaller number of subpools than the initial test pools by recombining the subpools of the selected test pools. The members of the second set of test pools are then tested for their ability to induce an immune response in vivo in a test animal such as a mouse, and the results of these tests are used to select one or more test pools from the second set of test pools which induce an immune response. This process is then repeated, each time reducing the number of subpools included in the test pools until a single subpool is selected. This subpool contains one (or at most a few) immunogenic mutant forms of the target antigen, which can be characterized by known technology (for example nucleic acid sequencing in the case of a nucleic acid antigen or peptide sequencing in the case of a peptide antigen).

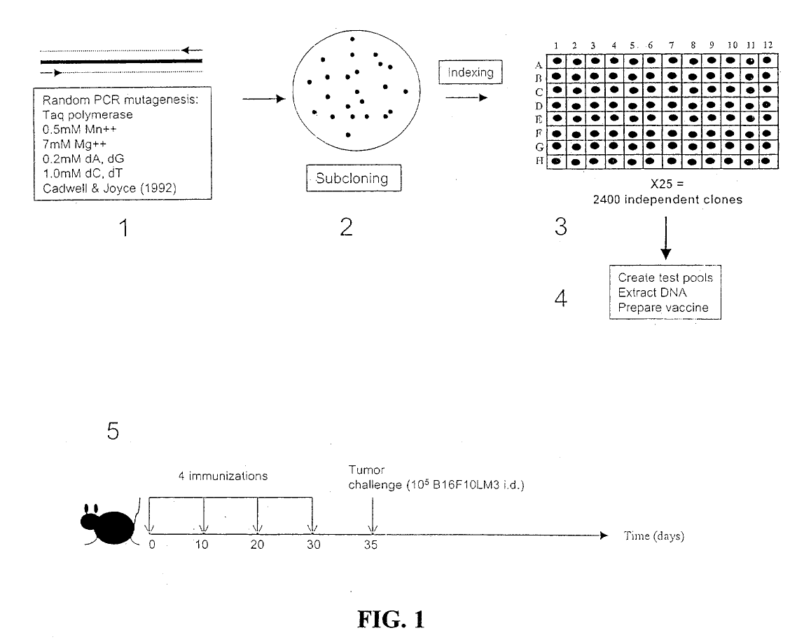

FIG. 1 illustrates the initial phase of a specific embodiment of the method of the invention in graphical form. In this example, mutations are created in the target DNA (1) and subcloned into a host organism, such a E. coli, which is grown on petri dishes (2). Colonies are picked from the petri dishes 1 and transferred to individual wells of plates (3). The plates are then incubated to grow the host organism within the wells. The contents of each well represents a subpool. Groups of subpools are them combined to create an initial set of test pools (4), and these are tested in test animals such as mice (5). The results of these tests are then used to guide the combination of the subpools into the second set of test pools.

The number of subpools created and combined into the initial set of test pools is not critical. It will be appreciated, however, that too small a number of subpools reduces the likelihood of identifying an immunogenic mutant form, while too large a number is cumbersome. In general a suitable number of subpools is on the order of 1000-10,000. The number of test pools (and the number of subpools per text pool) in the initial set of test pools is dependent on the number of subpools to be included in the test, and on convenience and economy since at least one test animal per test pool is required. In the example set forth below, 25 96-well plates were used to grow 2400 primary E. coli colonies. These plates were arranged in a 5×5 matrix, and each column and each row in the matrix was used to create one test pool in the initial set of test pools. Thus, for 2400 subpools, a total of 10 initial test pools were prepared, each containing 480 subpools. This experiment demonstrated that 2400 random mutations could be processed in only a few cycles of the invention to successfully identify mutant forms of inherently non-immunogenic antigens such as TRP-2 or gp75 that were immunogenic and which provided protection against tumors expressing these antigens.

When the mutant forms of the target antigen are nucleic acids, the nucleic acids in the subpool may be either DNA or RNA since both are known to useful in vaccine compositions. (See, for example, U.S. Pat. Nos. 5,580,859 and 5,589,466, Qiu et al., “Gene gun delivery of mRNA in situ results in efficient transgene expression and genetic immunization.” Gene Ther. 3(3):262-8 (1996); Garrity RR., “Targeted immune design using RNA immunization” Ann N Y Acad Sci.894:124-9 (1999); Zhou et al., “RNA melanoma vaccine: induction of antitumor immunity by human glycoprotein 100 mRNA immunization”, Hum Gene Ther. 10(16):2719-24 (1999); Giraud et al., “Generation of monoclonal antibodies to native human immunodeficiency virus type 1 envelope glycoprotein by immunization of mice with naked RNA”, J Virol Methods. 79(1):75-84 (1999); Dalemans et al., “Protection against homologous influenza challenge by genetic immunization with SFV-RNA encoding Flu-HA”, Ann N YAcad Sci. 772:255-6 (1995); Heiser et al., “Human dendritic cells transfected with RNA encoding prostate-specific antigen stimulate prostate-specific CTL responses In vitro”, J Immunol. 164(10):5508-14 (2000); Boczkowski et al., “Induction of tumor immunity and cytotoxic T lymphocyte responses using dendritic cells transfected with messenger RNA amplified from tumor cells”, Cancer Res.60(4):1028-34 (2000); Nair et al., “Induction of primary carcinoembryonic antigen (CEA)-specific cytotoxic T lymphocytes in vitro using human dendritic cells transfected with RNA”, Nat Biotechnol. 16(4):364-9 (1998); Ashley et al., “Bone marrow-generated dendritic cells pulsed with tumor extracts or tumor RNA induce antitumor immunity against central nervous system tumors”, J Exp Med. 186(7):1177-82 (1997), all of which are incorporated herein by reference).

Once the subpool containing a desirable mutant form of the target antigen is identified, the mutant form in that subpool is characterized and used in the making of a vaccine. Where the mutant form in the subpool is DNA, and the desired vaccine contains RNA, transcription can be carried out using alpha viruses or in vitro transcription systems. Once a mutant nucleic acid (DNA or RNA) is created, it is formulated into a vaccine composition for administration to the subject. As discussed below, one suitable mode of administration is subcutaneous injection of particles coated with the nucleic acid mixture using a GENE GUN. Thus, one embodiment of the vaccine composition comprises carrier particles coated with the pool of nucleic acid, i.e. with a mixture comprising a plurality of nucleic acid species encoding a plurality of mutant forms of the target antigen. The expressed mutant proteins or peptides are immunogenic and stimulate an immune response to the target antigen, even in the case where the target antigen is inherently non-immunogenic or only weakly immunogenic in the subject. The carrier particles used in this composition may be any of various types of particles known for use in this purpose, including without limitation gold, clay and tungsten. The particles suitably are from 0.5 to 3 microns in diameter to facilitate transdermal injection.

Other delivery systems which can be used to administer the nucleic acid vaccine compositions of the invention include the pressure delivery systems, for instance the BIOJECT system which delivers vaccines using carbon dioxide pressure cartridges. In this case, particles are not required, but can be used. The vaccine compositions can also be administered without a particle carrier using non-pressurized systems, for example syringe needles. Administration could also be accomplished using a mucosal route (e.g, a nasal spray). The pool of mutated DNA may also be incorporated into a viral vector, which is then associated with particles for adminstration by the routes described above.

The vaccine composition above may be administered in a liquid carrier by subcutaneous injection. For use in a Gene Gun, however, the composition is suitably packaged into therapeutic administration units, sometimes referred to as “bullets”. This is accomplished by drawing the composition into the lumen, a thin hollow tube, and then cutting the tube into lengths containing about 1 μg of nucleic acid.

In a second embodiment of the invention, the vaccine is a peptide vaccine created by expressing the antigen in the host organism prior to administration. Expression is suitably carried out in host cells which may be bacterial or eukaryotic (for example, yeast, insect or mammalian). For such expression, the mutated pool of nucleic acids are incorporated into an expression vector compatible with the host cells and then introduced into the host cells for expression in the colonies picked in the first step of the process. Such expression systems are well known in the art. An expressed marker may be included in the expression vector to facilitate selection of colonies which in which expression of the inserted materials is occurring.

A peptide vaccine identified using the method of the invention can be administered using methods known in the art, including without limitation by intravenous, intramuscular and subcutaneous injection and by transdermal or intranasal administration. The determination of the appropriate amount of peptide to vaccine to administer to arrive at the desired immune response is a routine matter within the ordinary skill in the art.

The invention will now be further described with reference to the following, non-limiting examples.

### EXAMPLE 1

To prepare mutant DNA, full length murine tyrosine-related protein 2 (mTRP-2) and murine gp75 were randomly mutated by PCR using the protocol of Cadwell et al., supra. Briefly, 20 ng of non-mutated plasmid encoding either protein served as a template for PCR. Mutagenic PCR was performed in Boehringer Mannheim's 1×PCR buffer supplemented to contain 7 mM MgCl2, 0.5 mM MnCl2, 0.2 mM dATP and dGTP, and 1 mM dCTP and dTTP. 30 PCR cycles were performed with primers pairs specific for each coding sequence (mTRP-2 primers,

| | | |
|---|---|---|
| upstream: | AAGGCGGCGCATGGGCCTTGTGGGATG, | (Seq. ID No. 1) |
| downstream: | ATGCGGCCGCTAGGCTTCCTCCGTGTA; | (Seq. ID No. 2) |
| mgp75 primers, upstream: | TTGCGGCCGCCATGAAATCTTACAACGTG, | (Seq. ID No. 3) |
| downstream: | CGGAATTCTCAGACCATGGAGTGGTTA | (Seq. ID No. 4)) |

and Taq polymerase from Boehringer Mannheim. The primers contained unique restriction sites for subsequent subcloning. Nonmutagenic control reactions were performed with the same template and primers using Stratagene's Pfu Turbo DNA polymerase in the corresponding buffer.

To generate pools of mutants, mutated and unmutated PCR products were subcloned into the CMV-based plasmid expression vector WRGBEN. Ross et al., Clin. Can. Res. 3: 2191-2196 (1997). The clones deriving from mutagenic PCR were plated and grown, so as to obtain pools of approximately 2,500 clones. The plasmid DNA of these clones was purified using QIAGEN 500 maxiprep columns in batches of variant plasmids containing random mutations. Each batch is referred to as a pool.

To assess the efficiency of PCR mutagenesis, four mutagenized clones of both mgp75 and mTRP-2 were sequenced over the first 600 bp of the coding sequence. Tables 1 and 2 list the observed mutations by type and extrapolate the numbers to the full-length coding sequences for mgp75 mutagenesis and mTRP-2 mutagenesis, respectively. The overall mutation frequency observed after 30 PCR cycles is 1.2 per 100 bp for mgp75, and 0.8 per 100 bp for mTRP-2.

TABLE 1
Random mutations in mgp75

| | | | Clone | | | | | | | | | | | | | | |
|---|---|---|---|---|---|---|---|---|---|---|---|---|---|---|---|---|---|
| | percent total | Total | 1 | 2 | 3 | 4 | 5 | 6 | 7 | 8 | 9 | 10 | 11 | 12 | 13 | 14 | 15 |
| Transversions | | | | | | | | | | | | | | | | | |
| A to T | 19.6 | 58 | 5 | 3 | 8 | 2 | 5 | 2 | 3 | 1 | 4 | 5 | 7 | 1 | 3 | 6 | 3 |
| A to C | 4.1 | 12 | 0 | 0 | 2 | 2 | 1 | 0 | 2 | 2 | 0 | 0 | 2 | 0 | 0 | 1 | 0 |
| G to T | 1.4 | 4 | 0 | 0 | 0 | 0 | 1 | 0 | 0 | 0 | 1 | 0 | 2 | 0 | 0 | 0 | 0 |
| G to C | 0.3 | 1 | 0 | 0 | 0 | 0 | 0 | 0 | 0 | 0 | 0 | 0 | 1 | 0 | 0 | 0 | 0 |
| T to A | 19.6 | 58 | 2 | 3 | 2 | 7 | 5 | 2 | 4 | 4 | 3 | 8 | 6 | 2 | 4 | 3 | 3 |
| T to G | 4.4 | 13 | 1 | 1 | 0 | 1 | 2 | 0 | 1 | 0 | 4 | 1 | 1 | 1 | 0 | 0 | 0 |
| C to A | 2.7 | 8 | 0 | 0 | 1 | 1 | 0 | 0 | 0 | 0 | 1 | 1 | 2 | 0 | 0 | 1 | 1 |
| C to G | 1.0 | 3 | 0 | 0 | 0 | 0 | 0 | 0 | 0 | 0 | 1 | 1 | 0 | 0 | 1 | 0 | 0 |
| Transitions | | | | | | | | | | | | | | | | | |
| A to G | 23.0 | 68 | 3 | 4 | 6 | 4 | 5 | 5 | 3 | 6 | 5 | 7 | 6 | 2 | 3 | 5 | 4 |
| G to A | 2.7 | 8 | 2 | 0 | 1 | 1 | 2 | 0 | 1 | 0 | 0 | 0 | 1 | 0 | 0 | 0 | 0 |
| C to T | 2.4 | 7 | 0 | 0 | 2 | 0 | 0 | 1 | 1 | 0 | 0 | 1 | 1 | 0 | 0 | 0 | 1 |
| T to C | 18.9 | 56 | 2 | 2 | 3 | 7 | 2 | 3 | 9 | 2 | 2 | 3 | 11 | 2 | 2 | 5 | 1 |
| Total | | | 15 | 13 | 25 | 25 | 23 | 13 | 24 | 15 | 21 | 27 | 40 | 8 | 13 | 21 | 13 |
| Deletions | | | 0 | 0 | 3 | 0 | 0 | 0 | 4 | 0 | 2 | 3 | 0 | 0 | 0 | 0 | 0 |
| Silent | | | 4 | 6 | 5 | 8 | 3 | 3 | 9 | 5 | 5 | 2 | 6 | 3 | 2 | 3 | 3 |
| Missense | | | 11 | 7 | 17 | 17 | 19 | 8 | 15 | 10 | 14 | 24 | 30 | 4 | 9 | 18 | 9 |
| Nonsense | | | 0 | 0 | 1 | 0 | 1 | 2 | 0 | 0 | 1 | 0 | 1 | 1 | 1 | 0 | 1 |
| Grand total | | 296 | | | | | | | | | | | | | | | |

| | | | |
|---|---|---|---|
| Nucleotides sequenced (all clones combined) | 24195 | | |
| Overall frequency | 0.012 | | |
| Average/clone | 19.7 | {close oversize brace} | Not including deletions |
| Standard deviation | 8.08 | | |

TABLE 2
Randon Mutations in mTRP-2

| | | | Clone | | | | | | | | | | | | | | |
|---|---|---|---|---|---|---|---|---|---|---|---|---|---|---|---|---|---|
| | percent total | Total | 1 | 2 | 3 | 4 | 5 | 6 | 7 | 8 | 9 | 10 | 11 | 12 | 13 | 14 | 15 |
| Transversions | | | | | | | | | | | | | | | | | |
| A to T | 25.7 | 56 | 4 | 5 | 2 | 3 | 3 | 4 | 1 | 3 | 2 | 4 | 5 | 5 | 4 | 9 | 2 |
| A to C | 4.1 | 9 | 0 | 1 | 0 | 2 | 1 | 0 | 0 | 1 | 0 | 2 | 1 | 0 | 0 | 1 | 0 |
| G to T | 0.5 | 1 | 1 | 0 | 0 | 0 | 0 | 0 | 0 | 0 | 0 | 0 | 0 | 0 | 0 | 0 | 0 |
| G to C | 0.0 | 0 | 0 | 0 | 0 | 0 | 0 | 0 | 0 | 0 | 0 | 0 | 0 | 0 | 0 | 0 | 0 |
| T to A | 22.0 | 48 | 2 | 1 | 4 | 1 | 4 | 3 | 2 | 4 | 4 | 2 | 8 | 5 | 0 | 4 | 4 |
| T to G | 3.7 | 8 | 1 | 2 | 0 | 0 | 0 | 1 | 0 | 1 | 0 | 0 | 1 | 1 | 1 | 0 | 0 |
| C to A | 1.4 | 3 | 0 | 0 | 0 | 0 | 0 | 1 | 1 | 0 | 1 | 0 | 0 | 0 | 0 | 0 | 0 |
| C to G | 0.0 | 0 | 0 | 0 | 0 | 0 | 0 | 0 | 0 | 0 | 0 | 0 | 0 | 0 | 0 | 0 | 0 |
| Transitions | | | | | | | | | | | | | | | | | |
| A to G | 16.5 | 36 | 6 | 1 | 2 | 1 | 1 | 1 | 2 | 2 | 6 | 5 | 2 | 1 | 1 | 0 | 5 |
| G to A | 2.8 | 6 | 1 | 1 | 0 | 1 | 0 | 1 | 0 | 0 | 0 | 1 | 0 | 1 | 0 | 0 | 0 |
| C to T | 3.7 | 8 | 0 | 1 | 1 | 0 | 0 | 3 | 0 | 1 | 0 | 0 | 0 | 0 | 1 | 0 | 1 |
| T to C | 19.7 | 43 | 2 | 3 | 3 | 5 | 2 | 2 | 6 | 3 | 2 | 4 | 0 | 3 | 3 | 1 | 4 |
| Total | | | 17 | 15 | 12 | 13 | 11 | 16 | 12 | 15 | 15 | 18 | 17 | 16 | 10 | 15 | 16 |
| Deletions | 0 | 1 | 0 | 1 | 0 | 1 | 0 | 1 | 1 | 0 | 3 | 0 | 0 | 0 | 0 | | |
| Silent | 5 | 1 | 4 | 2 | 4 | 6 | 4 | 3 | 5 | 6 | 1 | 4 | 2 | 3 | 5 | | |
| Missense | 10 | 14 | 8 | 10 | 7 | 9 | 8 | 12 | 10 | 11 | 15 | 11 | 9 | 12 | 10 | | |
| Nonsense | 2 | 0 | 0 | 1 | 0 | 0 | 0 | 0 | 0 | 1 | 1 | 1 | 0 | 0 | 1 | | |
| Grand total | | 218 | | | | | | | | | | | | | | | |

| | | | |
|---|---|---|---|
| Nucleotides sequenced (all clones | 22756 | | |
| combined) | | | |
| Overall frequency | 0.0096 | {close oversize parenthesis} | Not including deletions |
| Average/clone | 14.8 | | |
| Standard Deviat | 2.38 | | |

### EXAMPLE 2

Mutant forms of mTRP-2 were prepared as in Example 1. Mutated and unmutated PCR products were subcloned into the CMV-based plasmid expression vector WRGBEN. Ross et al., Clin. Can. Res. 3: 2191-2196 (1997) which was introduced into E coli. The E. coli was grown on petri dishes containing a selective agar medium (to select for a marker includes in the plasmid) and a total of 2400 colonies were selected for each target antigen and transferred into 96-well plates (a total of 25 plates per antigen).

The plates were arranged in a 5×5 matrix, and aliquots of all clones in each column and in each row of the matrix were combined to form 10 initial test pools of 480 clones. Mice were immunized with the DNA from each test pool, and assessed for tumor rejection.

In the initial test, 10 of the 10 test pools showed the presence of immunogenic mutant forms of the mTRP-2 antigen. Because each test pool represented a column or a row of a matrix, this test allowed us to identify discrete coordinates in the matrix (corresponding in this case to discrete 96-well plates) which must contain immunogenic mutants of mTRP-2. In the next round of testing, a 96-well plate of interest was treated as a matrix exactly as before, leading to positive coordinates encompassing 4 to 6 discrete clones. In the third and final round, these clones were tested individually. In the final round, 20 clones were tested individually, with 4 being immunogenic, displaying slower tumor growth than when mice were unimmunized or immunized with wild-type mTRP-2.

### EXAMPLE 3

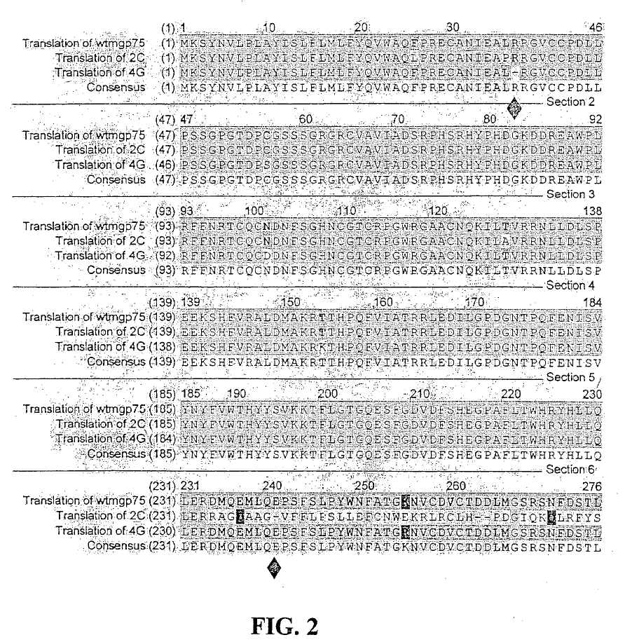

The experiment of Example 2 was repeated using mutagenized mgp75 DNA. Ultimately, 16 individual clones from 16 subpools were tested in the final step. Of these clones, 2 were found to be very immunogenic. Mutant “2C” confers tumor protection in 70% of immunized animals. Mutant “4G” confers 20% tumor protection but induces autoimmunity in 90% of immunized mice. Autoimmunity is displayed by loss of coat color. Under these same circumstances, treatment with wild-type mgp75 resulted in no tumor free mice, and treatment with human gp75 resulted in only 1 tumor free mouse out of 5. FIG. 2 shows the amino acid sequences of mutants “2C” and “4G” compared to the wild-type form. The diamonds indicate stop codons in the amino acid sequences of the mutants.

### EXAMPLE 4

Murine and human TRP-1 cDNAs were subcloned into the WRG7077BEN eukaryotic expression vector [Ross, 1997]. All PCR products encoding mutated or wt mTYRP-1 were generated using the following primers:

| | |
|---|---|
| 5′TTGCGGCCGCCATGAAATCTTACAACGTG3′; | (Seq. ID. No. 3) |
| 5′CGGAATTCTCAGACCATGGAGTGGTTA3′. | (Seq. ID. No. 4) |

The NotI and EcoRI restriction sites (underlined) were used for subcloning. Human DCT (TRP-2) cDNA was subcloned as an EcoRI fragment from the pCR3-hDCT plasmid (kindly provided by Dr. N. Restifo, National Cancer Institute, Bethesda, Md.) into WRG7077BEN.PCR products encoding mutated or wt mDCT were generated using the following primers:

| | |
|---|---|
| 5′AAGGCGCGCCATGGGCCTTGTGGGATG3′; | (Seq. ID. No. 1) |
| 5′ATGCGGCCGCTAGGCTTCCTCCGTGTA3′. | (Seq. ID. No. 2) |

They were subcloned in the WRGANEB variant of the pWRG7077BEN vector, using the AscI and NotI sites (underlined). This variant vector was created by inserting an oligonucleotide duplex in the NotI site of the WRG7077BEN vector. The partner oligonucleotides were:

(Seq. ID. No. 5)

5′GGCCTGGCGCGCCGTACGTTAACATCGATGC3′;

((Seq. ID. No. 6)

5′GGCCGCATCGATGTTAACGTACGGCGCGCCA3′.

To characterize discrete mutations, wt mDCT and mDCT mutants were subcloned into a derivative of pCR3 (Invitrogen, Carlsbad, Calif.), which was created by inserting the above duplex in the NotI site of the vector. The truncated cDNAs inserts for wt and M1 mDCT were created by PCR, using the same upstream oligonucleotide and a downstream oligonucleotide (5′ATGCGGCCGCTAGGCATTGGTCCCATTCAGGAAG3′ (Seq. ID. No. 7)), which covers the in-frame stop codon of M1 mDCT and contains a NotI site for cloning (underlined). Wt and M1 mDCT tagged at the C-terminus with the FLAG epitope were constructed by cloning the entire ORF of either variant between the EcoRV and SalI sites of pCMV-Tag 4A vector (Stratagene, La Jolla, Calif.). These fragments were reamplified with the following downstream primers, which abrogate the original stop codons of both variants to allow the in-frame fusion of the FLAG epitope coding sequence: wt mDCT,

| | | |
|---|---|---|
| mDCT, | 5′ATTCATAGTCGACGGCTTCCTCCGTGTATCTCTTG3′; | (Seq. ID. No. 8) |
| M1 mDCT, | 5′ATTCATAGTCGACGGCATTGGTCCCATTCAGGAAG3′. | (Seq. ID. No. 9) |

Correct inserts were checked by sequencing the entire ORFs.

For random mutagenesis of mTYRP-1 and mDCT by PCR, we applied the conditions reported by Cadwell and Joyce [Cadwell, 1994]. Briefly, the PCR mix contained 0.5 mM MnCl₂, 7 mM MgCl₂, 0.2 mM dATP, 0.2 mM dGTP, 1.0 mM dCTP, and 1.0 mM dTTP. We used 30 ng of plasmid template and Taq DNA polymerase (Roche, Indianapolis, Ind.) with the accompanying buffer.

Independent E. coli transformants containing subcloned mutagenized PCR products were plated at an approximate density of 800-1000 colonies per 100-mm agar plate. Either an indexed library protocol or a batch protocol was implemented. Library protocol: 2,400 clones were individually picked and arrayed on 96-well agar plates, which were replicated using a Boekel replicator tool (Fisher Scientific, Pittsburgh, Pa.). Colonies were replicated on agar dishes and regrown for DNA preparation. Batch protocol: each original 100 mm plate was replica-plated twice. In both protocols, colonies were then scraped off the plates and replicas, and were further grown in 300 ml antibiotic-supplemented Terrific Broth for 3-4 hours. Plasmid DNA was extracted using the Qiagen Maxi protocol.

1. A method for identifying a mutant form of a target antigen having immunogenic properties, comprising the steps of:
(a) creating a plurality of mutant forms of the target antigen;
(b) placing the mutant forms into a plurality of subpools, each subpool containing at least one mutant form of the target antigen;
(c) combining aliquots of the subpools into an initial set of test pools; each member of the initial of test pools comprising at least two of the subpools;
(d) testing each member of the initial set of test pools for the ability to induce an immune response to the target antigen in vivo;
(e) selecting at least one selected test pool from the initial set of test pools for further processing, said selected test pool being able to induce an immune response against the target antigen in vivo,
(f) combining further aliquots of the subpools which were included in the selected test pool to form a second set of test pools, each member of the second set if test pools comprising at least two of the subpools, and retesting each member of the second set of test pools for the ability to induce an immune response to the target antigen in vivo;
(g) performing additional cycles of selecting an effective pool from among the pools of a tested set of pools, and creating new sets of pools from the subpools making up that selected pool, until the selected pool contains only a single pool; and
(h) characterizing the mutant form of the antigen in the single pool.

2. The method of claim 1, wherein the mutant form of the antigen is a peptide.

3. The method of claim 1, wherein the mutant form of the antigen is a nucleic acid.

4. The method of claims 1, wherein the plurality of mutant forms of the target antigen are produced using error-prone PCR.

5. The method of claim 4, wherein the target antigen is selected from the group consisting of non-immunogenic or poorly immunogenic antigens.

6. The method of claim 5 wherein the target antigen is a self antigen of a subject to be treated.

7. The method of claim 2, wherein the target antigen is selected from the group consisting of non-immunogenic or poorly immunogenic antigens.

8. The method of claim 1, wherein the target antigen is selected from the group consisting of gp75/TRP-1, TRP-2, tyrosinase, gp100/pMel17, prostate specific membrane antigen, prostate specific antigen, prostate stem cell antigen, HER2/neu, the mucin MUC1, CD19, CD20, MAGE, BAGE, GAGE, NY-ESO-1 and other “cancer-testes” antigens, gene products from the human immunodeficiency virus-1; angiogenic factors, tumor suppressor genes, dipeptidyl peptidase IV and fibroblast activation protein-1.

9. The method of claim 8, wherein the mutant form of the antigen is a peptide.

10. The method of claim 8, wherein the mutant form of the antigen is a nucleic acid.

11. The method of claims 8, wherein the plurality of mutant forms of the target antigen are produced using error-prone PCR.

12. The method of claim 11, wherein the target antigen is selected from the group consisting of non-immunogenic or poorly immunogenic antigens.

13. The method of claim 12, wherein the target antigen is a self antigen of a subject to be treated.

14. A method for preparing a vaccine for inducing an immune response to a target antigen, comprising the steps of identifying a mutant form of the target antigen using a method, comprising the steps of:
(a) creating a plurality of mutant forms of the target antigen;
(b) placing the mutant forms into a plurality of subpools, each subpool containing at least one mutant form of the target antigen;
(c) combining aliquots of the subpools into an initial set of test pools; each member of the initial of test pools comprising at least two of the subpools;
(d) testing each member of the initial set of test pools for the ability to induce an immune response to the target antigen in vivo;
(e) selecting at least one selected test pool from the initial set of test pools for further processing, said selected test pool being able to induce an immune response against the target antigen in vivo,
(f) combining further aliquots of the subpools which were included in the selected test pool to form a second set of test pools, each member of the second set if test pools comprising at least two of the subpools, and retesting each member of the second set of test pools for the ability to induce an immune response to the target antigen in vivo;
(g) performing additional cycles of selecting an effective pool from among the pools of a tested set of pools, and creating new sets of pools from the subpools making up that selected pool, until the selected pool contains only a single pool; and
(h) characterizing the mutant form of the antigen in the single pool, and formulating the mutant form of the target antigen into a pharmaceutically acceptable vaccine composition.

15. The method of claim 14, wherein the mutant form of the antigen is a peptide.

16. The method of claim 14, wherein the mutant form of the antigen is a nucleic acid.

17. The method of claims 14, wherein the plurality of mutant forms of the target antigen are produced using error-prone PCR.

18. The method of claim 17, wherein the target antigen is selected from the group consisting of non-immunogenic or poorly immunogenic antigens.

19. The method of claim 18 wherein the target antigen is a self antigen of a subject to be treated.

20. The method of claim 14, wherein the target antigen is selected from the group consisting of gp75/TRP-1, TRP-2, tyrosinase, gp100/pMel17, prostate specific membrane antigen, prostate specific antigen, prostate stem cell antigen, HER2/neu, the mucin MUC1, CD19, CD20, MAGE, BAGE, GAGE, NY-ESO-1 and other “cancer-testes” antigens, gene products from the human immunodeficiency virus-1; angiogenic factors, tumor suppressor genes, dipeptidyl peptidase IV and fibroblast activation protein-1.
